(19) United States
(12) Patent Application Publication
(10) Pub. No.: US 2016/0199600 A1
(43) Pub. Date: Jul. 14, 2016

(54) SINGLE USE INTRANASAL ATOMIZER

(71) Applicant: BIXA RESEARCH AND HEALTH INC., Vancouver (CA)

(72) Inventor: Amin JANMOHAMED, Vancouver (CA)

(73) Assignee: BIXA RESEARCH AND HEALTH INC., Vancouver (CA)

(21) Appl. No.: 14/916,554

(22) PCT Filed: Jun. 4, 2014
(86) PCT No.: PCT/CA2014/050513

§ 371 Date: Mar. 3, 2016

Related U.S. Application Data
(60) Provisional application No. 61/876,642, filed on Sep. 11, 2013.

(51) Int. Cl. A61M 15/08; A61M 15/00; A61M 11/00

(52) U.S. Cl. CPC A61M 15/08; A61M 11/007; A61M 15/0025

(57) ABSTRACT

An intranasal atomizer drug chamber housing comprises an atomizable liquid, an atomizer chamber; a slidable plunger; an atomizer in the atomizer chamber and a spring to drive the plunger upon release from a compressed position. The intranasal atomizer may additionally comprise: a drug chamber veil attached to outlet end of the atomizer, and shaped to fit in a user's nostril. A trigger housing comprising a trigger button and a trigger mechanism may be attached to the drug chamber housing. The trigger mechanism retains the spring in a compressed position, and releases the spring to drive the plunger upon activation of the trigger button. A safety cap fits over the outlet end of the drug chamber housing and has a flexible arm to insert within the trigger mechanism to prevent the release of the spring. A method of providing an atomisable drug or medicament using the intranasal atomizer is also disclosed.

### CROSS-REFERENCE TO RELATED APPLICATIONS

This application is related and claims priority to U.S. Provisional Patent Application Ser. No. 61/876,642 filed Sep. 11, 2013, which is hereby incorporated herein by reference in its entirety.

### TECHNICAL FIELD

The present invention relates to atomizers, and more particularly relates to intranasal atomizers by which drugs or medicine may be sprayed into the nostril of a patient.

### BACKGROUND OF THE INVENTION

Nasal atomizers have long been used to deliver drugs or medicaments to a patient. Intranasal administration of drugs or other medical substances is useful since it is non-invasive. Medicaments are absorbed readily through the nasal mucous membranes, so the need for injection by a syringe is removed and the procedure is less painful for the patient. One problem with intranasal drug delivery is achieving an accurately measured dosage. Another problem with existing nasal atomizer designs is that if they are re-used by different patients there is a danger of transmitting disease between successive users. Where a squeeze bottle is used, the drug which has been contaminated may be sucked back into the dispenser and contaminated drugs re-delivered to the next user.

A current form of nasal drug delivery device is the MAD mucosal atomization device which is an adaptor which attaches to a syringe. The drug to be administered is drawn into the syringe and the atomizer cap is attached to the syringe. The atomizer is placed in the nostril and the plunger of the syringe is depressed. The appropriate volume of the drug needs to be measured into the syringe and proper pressure applied to the plunger to obtain the right atomization. This is difficult to achieve in delivering small volume of drug solution, where there is a larger dead space in the syringe. It is also difficult for the user to provide a consistent, precise actuation force on the plunger.

If used with a pre-filled drug syringe, the device is prone to leakage since there is no closure feature.

Other nasal drug delivery devices are the Zomig™ nasal spray manufactured by AstraZeneca and the Imitrex™ nasal spray manufactured by GlaxoSmithKline which use plungers. The factors which determine characteristics or properties of the spray delivered into the nostril of a patient are the viscosity of the solution, size of the nasal aperture and turbinates and amount of force applied in pushing the liquid through the turbinates and aperture. The force applied in actuating Zomig and Imitrex is based on resistance at the base of the plunger. One must push with some force to break through the restriction at the plunger which causes the user to apply minimal force. Such devices therefore lack consistency in use from one user to another as someone could push harder on the plunger than is required. This affects the spray characteristics. Neither device has a dose indicator. Dose confirmation is obtained just from noticing a loose plunger or flange around the plunger.

There is therefore a need for a single use, intranasal atomizer that uses a consistent activation force for atomization, delivers an accurate dosage and cannot be re-used.

The foregoing examples of the related art and limitations related thereto are intended to be illustrative and not exclusive. Other limitations of the related art will become apparent to those of skill in the art upon a reading of the specification and a study of the drawings.

### SUMMARY OF THE INVENTION

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

The present invention provides a single use intranasal atomizer that has a consistent activation force for atomization, delivers an accurate dosage and cannot be re-used by providing a spring-loaded delivery mechanism that cannot be re-loaded by the patient. More particularly, the invention provides a spring-driven plunger which is trigger-released and forces the liquid medicament from a drug storage chamber by means of a piston and out of the dispensing tip of the device through an atomizer. Once the trigger has been fired, the device cannot be reloaded or re-used. Preferably the drug chamber is filled with the dosage of the drug and hermetically sealed by a safety cap prior to assembly of the device and loading of the trigger.

In one embodiment of the present invention, an intranasal atomizer is provided, comprising: a drug chamber housing comprising a hollow drug chamber operable to contain an atomizable liquid and an outlet end comprising an atomizer chamber; a plunger sealingly situated within the drug chamber and slidable within the drug chamber; an atomizer situated at least partially within the atomizer chamber at the outlet end of the housing; and a spring operable to drive the plunger within the drug chamber upon release from a compressed position. In a further such embodiment, the intranasal atomizer additionally comprises: a drug chamber veil attached to the drug chamber housing, the veil comprising a smoothly contoured outer surface operable to fit within a nostril of a user, and a hollow central bore; wherein the outlet end of the drug chamber housing fits within the hollow central bore of the veil.

According to another embodiment of the present invention, the intranasal atomizer described above may additionally comprise: a trigger housing comprising a hollow bore and a trigger button on an outer surface of the trigger housing; and a trigger mechanism situated within the hollow bore of said trigger housing; wherein the trigger housing is attached to a base flange situated at a rear end of the drug chamber housing opposite the outlet end, and the trigger mechanism is operable to retain the spring in a compressed position, and to release the spring to drive the plunger upon activation of the trigger button. In yet another embodiment, the intranasal atomizer may also comprise a safety cap comprising: a closed front end, a hollow interior adapted to fit over the outlet end of the drug chamber housing and a flexible arm extending rearward away from the front end and operable to insert within the trigger mechanism to prevent the release of the spring when the safety cap is in place over the outlet end.

In a further embodiment according to the present invention, a method of providing an atomisable drug or medicament is provided. A first step of the method comprises providing a sealed drug chamber housing comprising: a predetermined dose of an atomisable liquid drug or medicament in a hollow drug chamber of the housing; an atomizer situated at an outlet end of the housing and in fluid communication with the hollow drug chamber; a plunger sealingly situated within the hollow drug chamber and slidable within the drug chamber; and a spring operable to drive the plunger within the drug chamber upon release from a compressed position. The method also comprises providing a safety cap attached to the outlet end of the drug chamber housing; and providing a trigger housing comprising: a trigger button and a trigger mechanism attached to a base flange of the drug chamber housing, wherein the trigger mechanism is operable to retain the spring in a compressed position and to release the spring to drive the plunger upon activation of the trigger button. In yet a further embodiment, the method of providing an atomisable drug or medicament additionally comprises: removing the safety cap; placing the outlet end of the drug chamber housing proximate to a nostril of a user; and activating the trigger button to release the spring and drive the plunger, delivering the atomisable drug or medicament into the nostril of the user.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is a perspective view of an intranasal atomizer according to an embodiment of the invention.

FIG. 2 is a top plan view of an intranasal atomizer according to an embodiment of the invention.

FIG. 3 is a right side elevation view of an intranasal atomizer according to an embodiment of the invention.

FIG. 4 is a left side elevation view of an intranasal atomizer according to an embodiment of the invention.

FIG. 5 is a bottom plan view of an intranasal atomizer according to an embodiment of the invention.

FIG. 6 is an end view of an intranasal atomizer according to an embodiment of the invention.

FIG. 7 is a cross-sectional view of an intranasal atomizer according to an embodiment of the invention, taken along lines 7-7 of FIG. 1.

FIG. 8 is a perspective cross-sectional view of an intranasal atomizer according to an embodiment of the invention, taken along lines 7-7 of FIG. 1.

FIG. 9 and FIG. 10 are exploded views of an intranasal atomizer according to an embodiment of the invention.

FIG. 11 is a partial exploded view of the drug chamber assembly of an intranasal atomizer according to an embodiment of the invention.

FIG. 12 is a partial exploded view of the trigger construction of an intranasal atomizer according to an embodiment of the invention.

FIGS. 13a, 13b, 13c and 13d are sequential perspective views illustrating a drug discharge from an intranasal atomizer according to an embodiment of the invention.

FIGS. 14a, 14b and 14c are sequential cross-sectional views illustrating the interaction of a trigger and a safety cap of an intranasal atomizer according to an embodiment of the invention.

FIG. 15 is a cross-sectional view of a drug chamber/atomizer housing of an intranasal atomizer according to an embodiment of the invention.

FIG. 16 is a detail perspective view of the atomizer of an intranasal atomizer according to an embodiment of the invention.

FIG. 17 is an end view of the atomizer of an intranasal atomizer according to an embodiment of the invention.

FIG. 18 is an end view of the safety cap of an intranasal atomizer according to an embodiment of the invention.

FIG. 19 is a detail view of the break-away tabs securing the trigger button of an intranasal atomizer according to an embodiment of the invention.

### DETAILED DESCRIPTION OF SEVERAL EMBODIMENTS

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

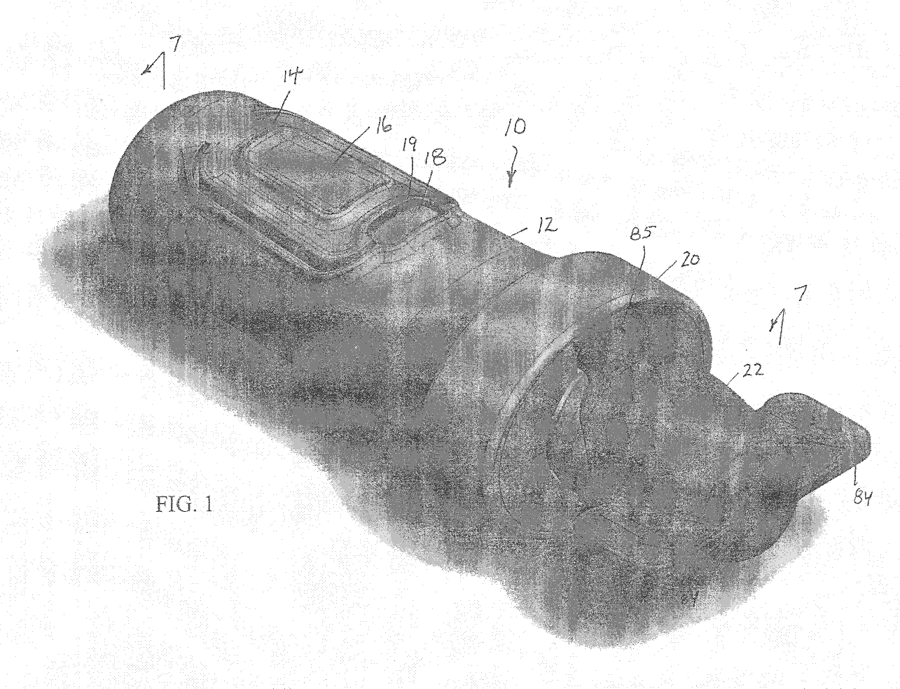

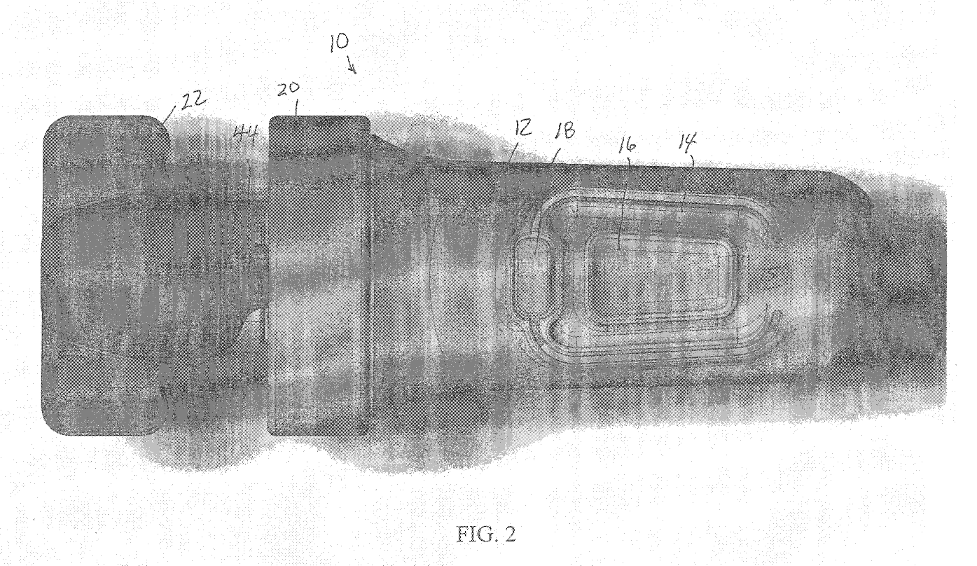

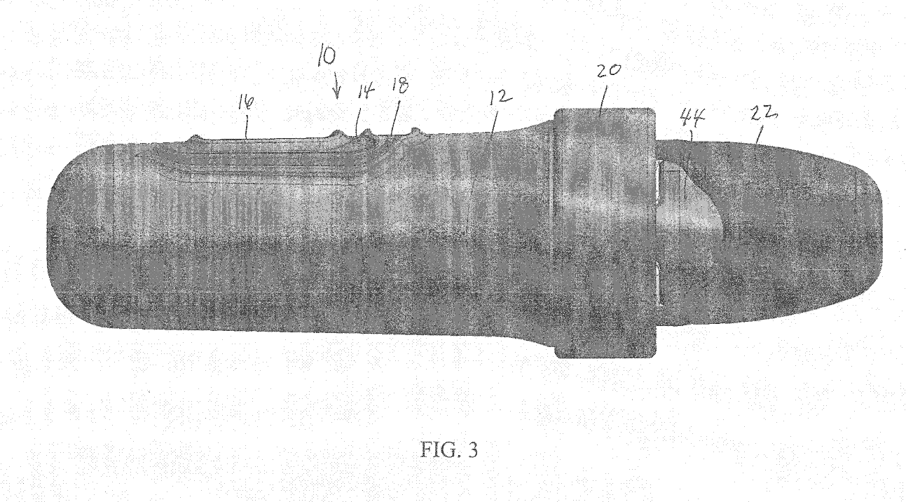

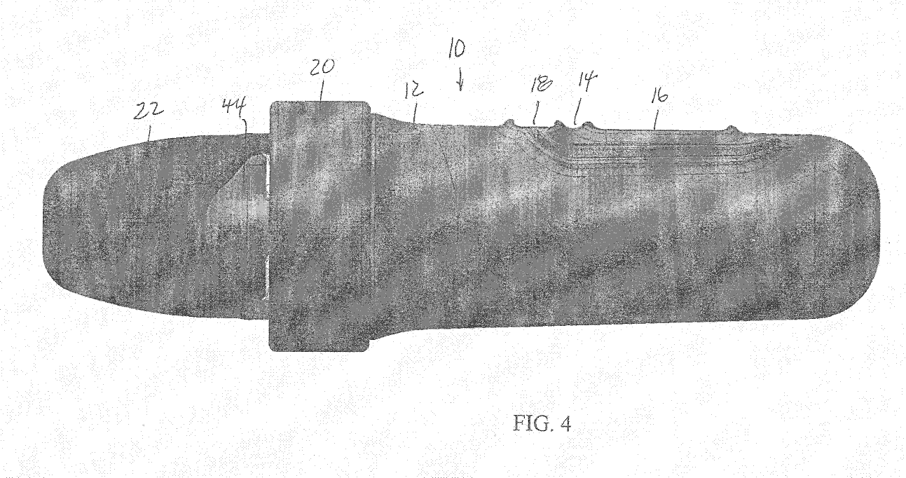

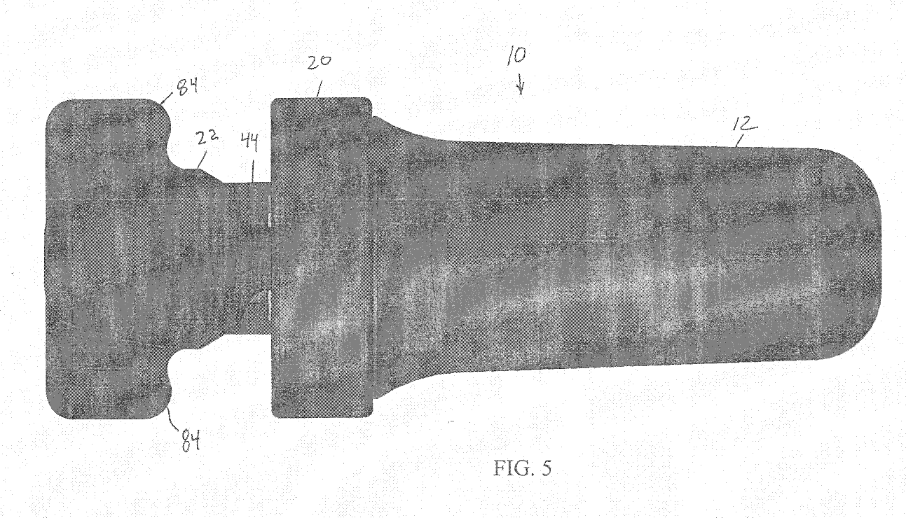

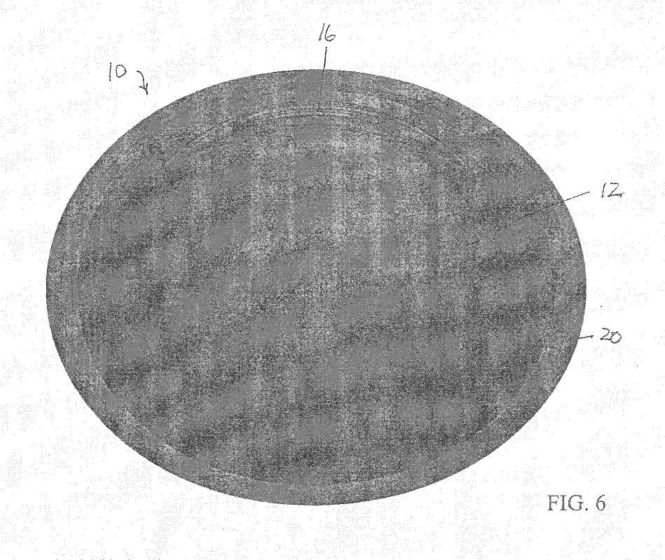

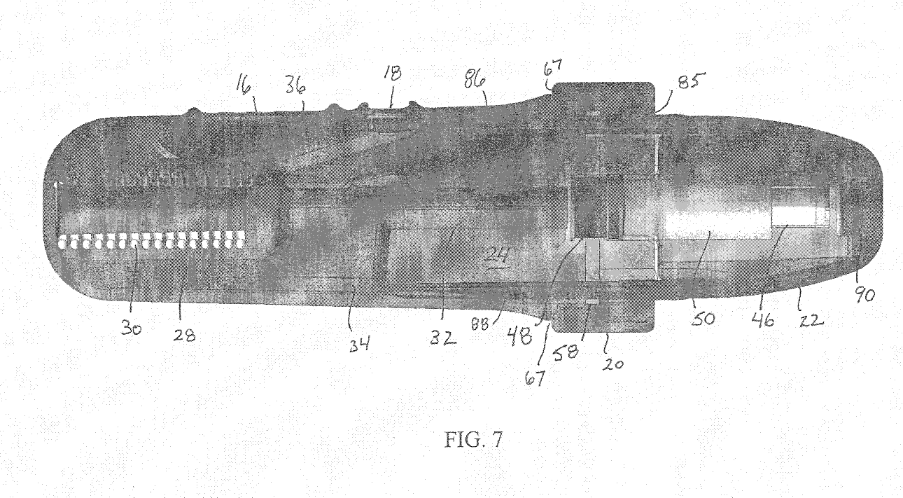

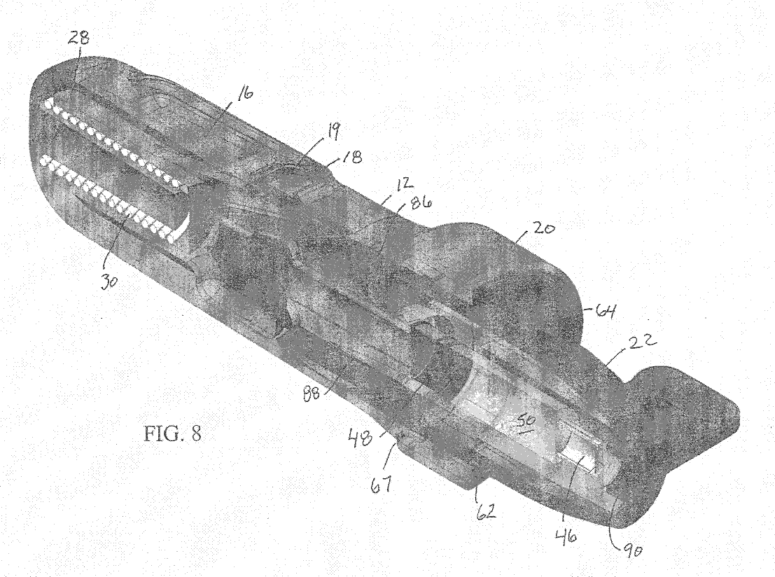

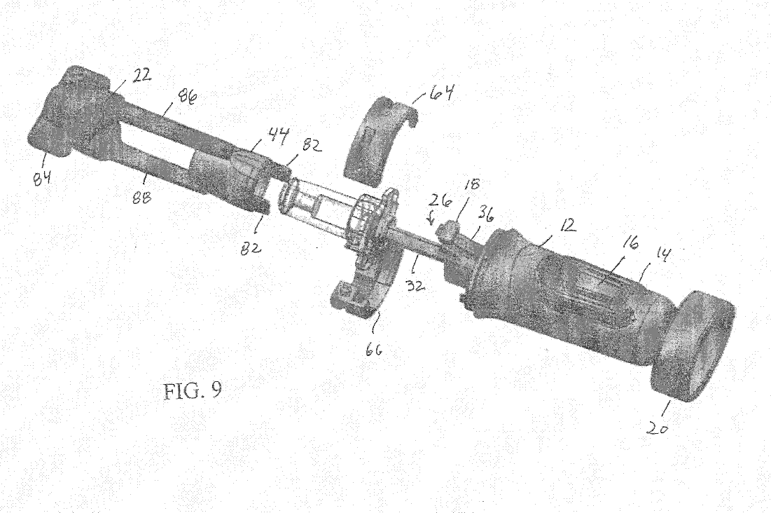

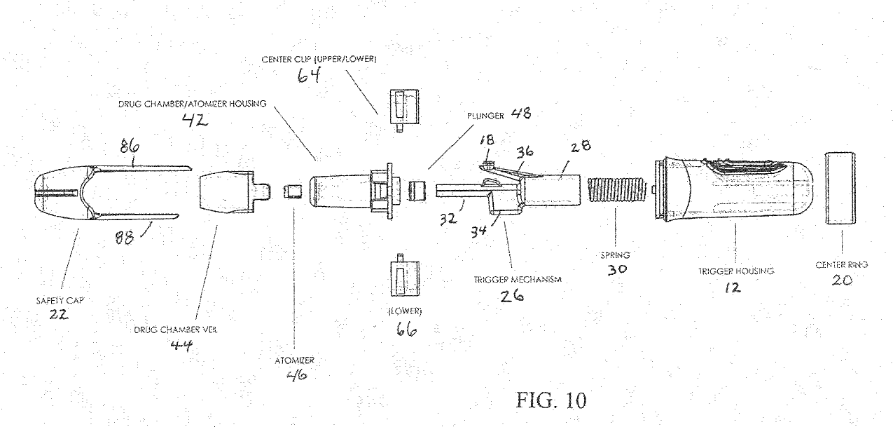

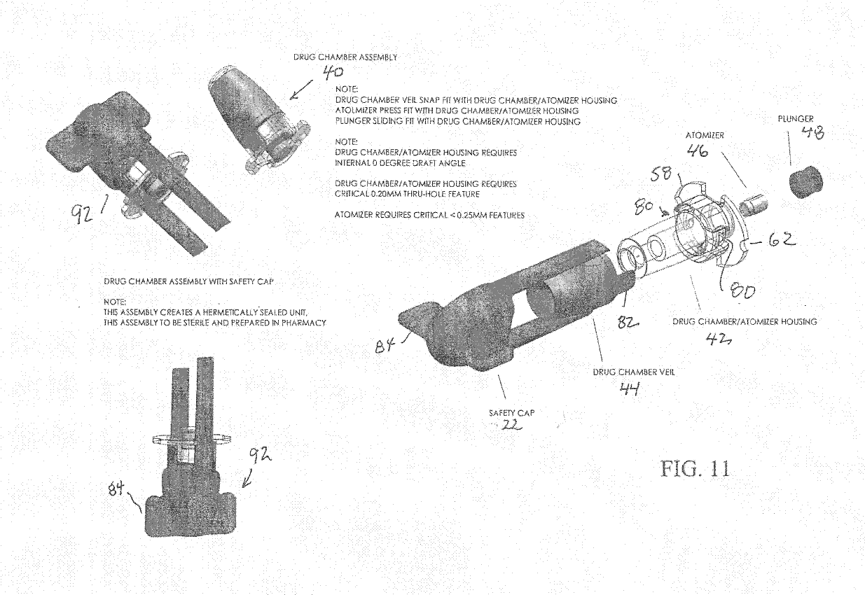

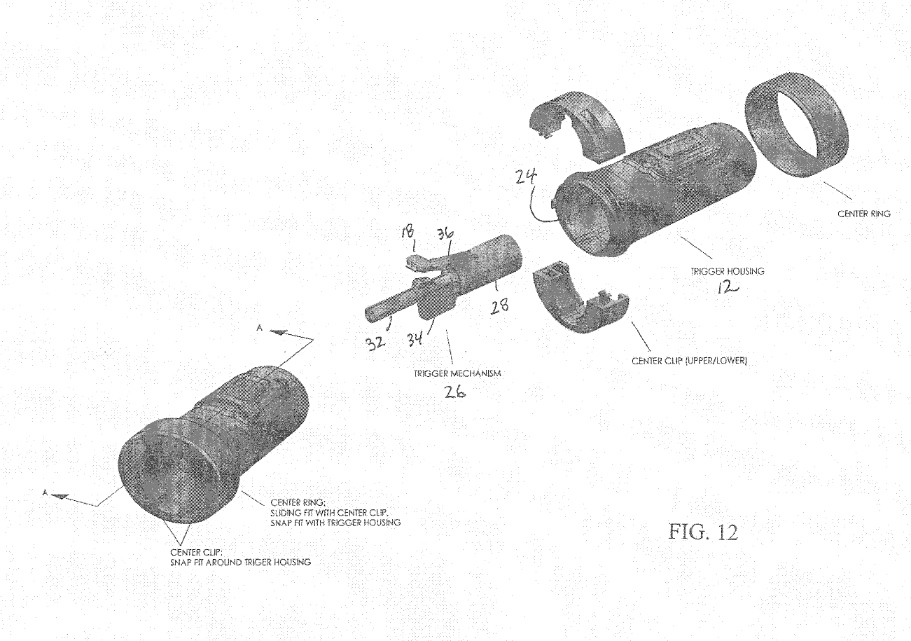

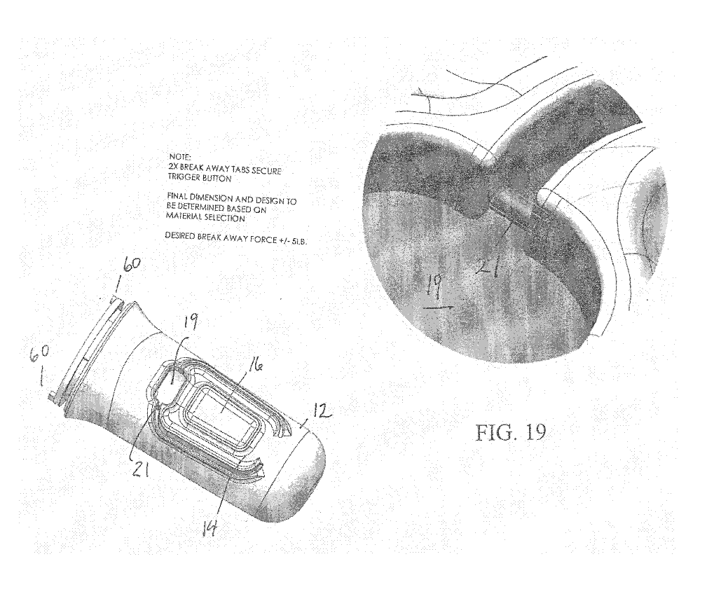

With reference to FIG. 1-10, the intranasal atomizer 10 has a trigger housing 12 with hinged trigger panel 14, trigger button 16, trigger indicator aperture 19, centre ring 20 and safety cap 22. Trigger housing 12 forms a hollow chamber 24 which receives trigger mechanism 26. Trigger mechanism 26 has a hollow cylindrical base 28 for receiving spring 30, cylindrical piston 32, guide body 34 and flexible firing pin 36 at the free end of which is the trigger indicator panel 18. To prevent unintended triggering of the device, trigger panel 14 is secured by break-away tabs 21 (FIG. 19) until sufficient pressure is applied to trigger button 16.

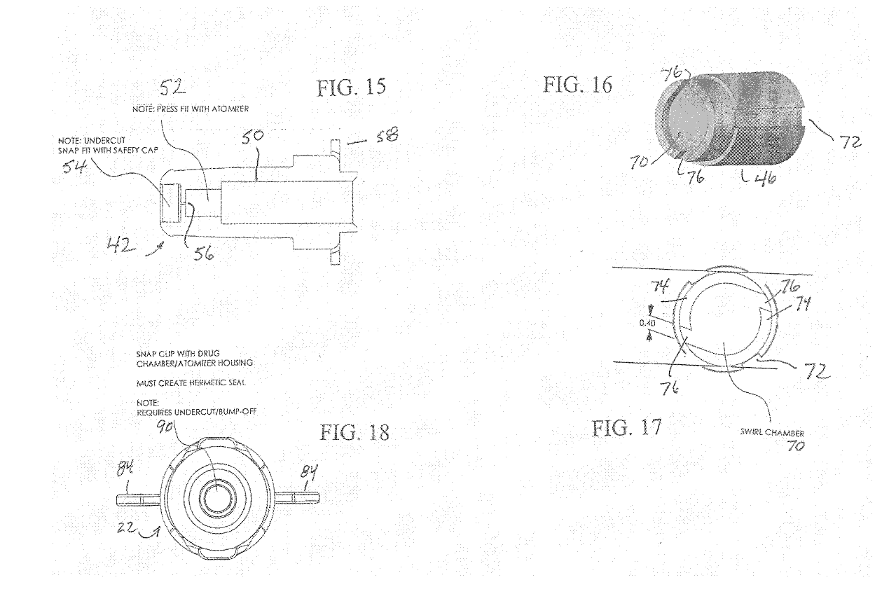

Drug chamber assembly 40 (FIG. 11) includes drug chamber/atomizer housing 42, drug chamber veil 44, atomizer 46 and plunger 48. Drug chamber veil 44 has an external smoothly contoured shape to fit into a user's nostril. Drug chamber/atomizer housing 42 has a hollow central cylindrical chamber 50 which holds the dosage of the drug to be administered. Chamber 50 communicates with cylindrical chamber 52 which seats the atomizer 46. Cylindrical chamber 52 (FIG. 15) communicates with an external cylindrical opening 54 through a passage 56. Drug chamber/atomizer housing 42 has a base flange 58 by means of which drug chamber assembly 40 is secured to the trigger housing 12. Protrusions 60 on the trigger housing 12 engage with openings 62 on flange 58. Upper and lower center clips 64, 66 are brought together to secure flange 58 against trigger housing 12, snap together and then are tightly secured by the center ring 20 which has a sliding fit with center clips 64, 66 and locks with an interference clip at 67 to the trigger housing 12.

Atomizer 46 has a swirl chamber 70 on the downstream end thereof. The drug is delivered along longitudinal passages 72 and then travels circumferentially around cylindrical walls 74, entering the swirl chamber through passages 76. The outer surface 78 of the atomizer press fits into chamber 52. After the atomizer has been installed, the chamber 50 is filled with the measured dosage of the drug, and plunger 48 is installed to retain the liquid in chamber 50. Plunger 48 has a sealing slide fit in chamber 50. To complete the drug chamber assembly 40, the drug chamber veil 44 has tabs 82 which snap fit into slots 80 in drug chamber/atomizer housing 42.

Safety cap 22 has ears 84 to assist the user in removing the cap, upper flexible arm 86 which engages the firing pin 36 and lower guide arm 88 and engagement cylinder 90 which sealingly snap fits into external cylindrical opening 54 in the drug chamber/atomizer housing 42. In this way the drug chamber assembly is hermetically sealed after it has been loaded with the drug dosage.

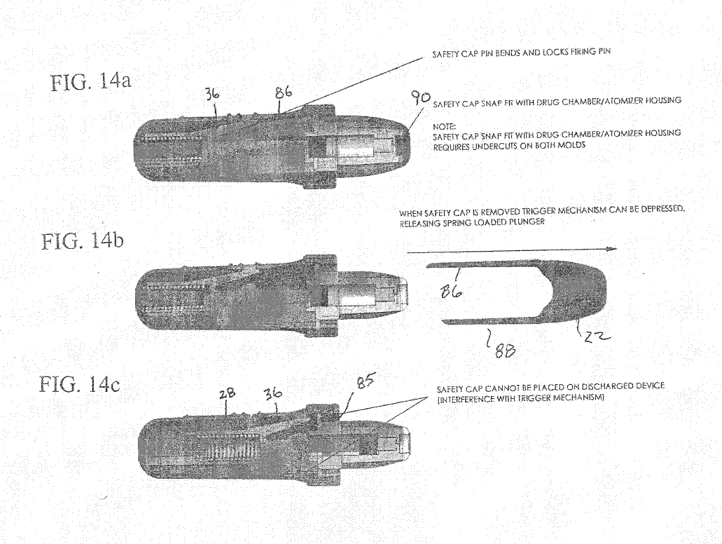

As shown in FIG. 14a, when the safety cap 22 is installed in place, arm 86 bends under the firing pin 36. As shown in FIG. 14b, when the safety cap 22 is removed, the indicator 18 remains locked in opening 19 in the trigger housing to retain the firing pin 36 in place against the pressure of spring. When the trigger button 16 is pressed, hinged trigger panel 14 pivots about hinge area 15 and presses firing pin 36 downwardly, releasing indicator 18 from aperture 19 and permitting spring 30 to force trigger mechanism 26 forwardly to the position shown in FIG. 14c. Once in that forward position, the firing pin 36 blocks aperture 85 from permitting the safety cap arm 86 to be re-inserted. Thus the safety cap 22 cannot be replaced on a discharged device nor can the device be discharged again.

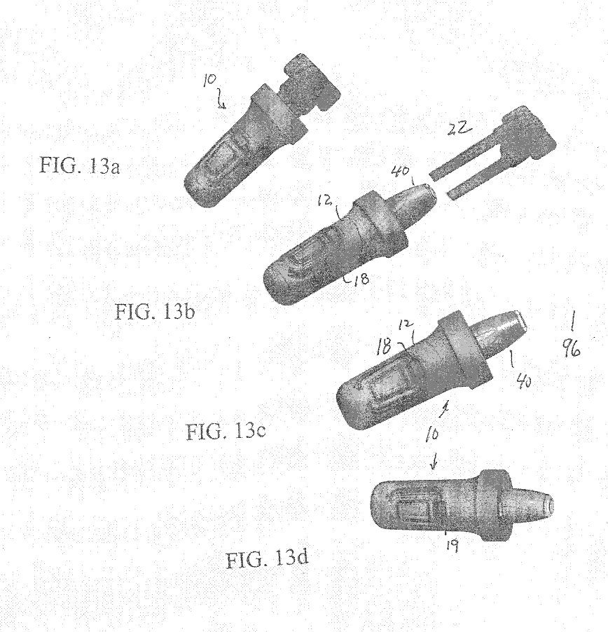

To assemble a loaded device 10, the drug chamber assembly 40 and safety cap 22 are assembled in a sterile environment to create a hermetically sealed unit 92 as shown in FIG. 11. Drug chamber veil 44 and safety cap 22 are installed over drug chamber and atomizer housing 42 so that opening 54 is hermetically sealed by engagement cylinder 90. Atomizer 46 is installed into chamber 52, the drug 96 is loaded into chamber 50 and sealed in place by plunger 48. Trigger assembly 26 is then locked into place in housing 12, compressing spring 30 with the firing pin secured in aperture 19 to hold the trigger assembly 26 in the loaded position. Assembly 92 can then be secured to the trigger housing assembly as shown in FIG. 14a, with the safety cap arm locking the firing pin 36 in place and resulting in the loaded, locked assembly as shown in FIG. 13a.

To deliver the drug, the user removes the safety cap 22 (FIG. 13b), presses button 16 which breaks tabs 21 and causes the trigger assembly to fire by spring 30 driving rod 32 against plunger 48, forcing the drug 96 through the atomizer 46 and out passage 56 and into the user's nostril (FIG. 13c). Once the drug has been thereby discharged the atomizer 10 cannot be re-used since there is no access to the user to re-load the spring and trigger assembly. Once used, the entire unit 10 is then disposed of to avoid contamination or infection of a subsequent user.

The indicator aperture 19 may show a different appearance after discharge to indicate to the user that the device has been used. For example, the indicator panel 18 may be coloured green when loaded and prior to discharge to show the device is ready to use but after discharge aperture 19 is coloured red for example (FIG. 13d). A simple way to achieve this is to have the trigger mechanism including indicator 18 molded in a green plastic with part of the base 28 colored red. When the device is loaded the section of the green indicator 18 is visible, and fills the indicator aperture 19. When the device is fired, the trigger mechanism moves forward and the base 28 of the trigger mechanism is visible through the indicator aperture 19. This section of the trigger mechanism 28 may be covered with a red vinyl sticker during assembly or manufacture of the trigger mechanism or the trigger mechanism can be over-molded, or molded in two parts to accomplish this coloration.

The size and strength of spring 30, and the length of travel of trigger mechanism 26 are selected in conjunction with the volume and viscosity of the drug dosage and size of the atomizer 46 outlet passages 72, 76 to provide the desired spray characteristics.

While a number of exemplary aspects and embodiments have been discussed above, those of skill in the art will recognize certain modifications, permutations, additions and sub-combinations thereof. It is therefore intended that the invention be interpreted to include all such modifications, permutations, additions and sub-combinations as are within their true spirit and scope.

Reference throughout this specification to “one embodiment,” “an embodiment,” or similar language means that a particular feature, structure, or characteristic that is described in connection with the embodiment is included in at least one embodiment of the present disclosure. Thus, appearances of the phrases “in one embodiment,” “in an embodiment,” and similar language throughout this specification may, but do not necessarily, all refer to the same embodiment. Further, the described features, structures, or characteristics of the present disclosure may be combined in any suitable manner in one or more embodiments. In this Detailed Description, numerous specific details are provided for a thorough understanding of embodiments of the disclosure. One skilled in the relevant art will recognize, however, that the embodiments of the present disclosure can be practiced without one or more of the specific details, or with other methods, components, materials, and so forth. In other instances, well-known structures, materials, or operations are not shown or described in detail to avoid obscuring aspects of the present disclosure.

The scope of the present disclosure fully encompasses other embodiments and is to be limited, accordingly, by nothing other than the appended claims, wherein any reference to an element being made in the singular is intended to mean “one or more”, and is not intended to mean “one and only one” unless explicitly so stated. All structural and functional equivalents to the elements of the above-described preferred embodiment and additional embodiments are hereby expressly incorporated by reference and are intended to be encompassed by the present claims. Moreover, no requirement exists for an apparatus or method to address each and every problem sought to be resolved by the present disclosure, for such to be encompassed by the present claims. Furthermore, no element, component, or method step in the present disclosure is intended to be dedicated to the public regardless of whether the element, component, or method step is explicitly recited in the claims. However, that various changes and modifications in form, material, work-piece, and fabrication material detail may be made, without departing from the spirit and scope of the present disclosure, as set forth in the appended claims, are also encompassed by the present disclosure.

What is claimed is:

1. An intranasal atomizer comprising:
a drug chamber housing comprising a hollow drug chamber operable to contain an atomizable liquid and an outlet end comprising an atomizer chamber;
a plunger sealingly situated within said drug chamber and slidable within said drug chamber;
an atomizer situated at least partially within said atomizer chamber at said outlet end of said housing; and
a spring operable to drive said plunger within said drug chamber upon release from a compressed position.

2. The intranasal atomizer according to claim 1 additionally comprising:
a drug chamber veil attached to said drug chamber housing, said veil comprising a smoothly contoured outer surface operable to fit within a nostril of a user, and a hollow central bore;
wherein said outlet end of said drug chamber housing fits within said hollow central bore of said veil.

3. The intranasal atomizer according to claim 1 additionally comprising:
a trigger housing comprising a hollow bore and a trigger button on an outer surface of said trigger housing; and
a trigger mechanism situated within said hollow bore of said trigger housing;
wherein said trigger housing is attached to a base flange situated at a rear end of said drug chamber housing opposite said outlet end, and said trigger mechanism is operable to retain said spring in a compressed position, and to release said spring to drive said plunger upon activation of said trigger button.

4. The intranasal atomizer according to claim 3 additionally comprising:
a safety cap comprising a closed front end, a hollow interior adapted to fit over said outlet end of said drug chamber housing and a flexible arm extending rearward away from said front end and operable to engage said trigger mechanism to prevent said release of said spring when said safety cap is in place over said outlet end.

5. The intranasal atomizer according to claim 3, wherein said trigger mechanism comprises a firing pin situated adjacent to said trigger button, and a piston situated between said spring and said plunger, and wherein displacement of said firing pin by said trigger button is operable to release said spring against said piston to drive said plunger.

6. The intranasal atomizer according to claim 4, wherein said safety cap comprises a second flexible arm extending away from said front end and operable to sealingly attach said safety cap to said drug chamber housing.

7. The intranasal atomizer according to claim 6, wherein said second flexible arm is additionally operable to lockingly engage said safety cap to an opening in a lower flange of said drug chamber housing.

8. The intranasal atomizer according to claim 1, wherein said hollow drug chamber comprises a liquid drug or medicament.

9. The intranasal atomizer according to claim 1, wherein said atomizer comprises a swirl chamber.

10. The intranasal atomizer according to claim 9, wherein said swirl chamber is situated at an outlet end of said atomizer, and wherein said atomizer additionally comprises longitudinal flow passages extending between said atomizer chamber and said swirl chamber.

11. The intranasal atomizer according to claim 1, wherein said atomizer is press fit into said atomizer chamber at said outlet end of said housing.

12. The intranasal atomizer according to claim 3, wherein said trigger housing additionally comprises a center ring situated at a forward end thereof, and said drug chamber housing comprises a base flange at a rear end opposite said outlet end, and wherein said center ring is operable to attach said trigger housing to said base flange of said drug chamber housing.

13. The intranasal atomizer according to claim 3, wherein said trigger housing comprises a hollow cylindrical base at a rearward end thereof, and said base is adapted to receive said spring.

14. The intranasal atomizer according to claim 5, wherein said firing pin comprises a trigger indicator at a free end thereof, and wherein said trigger indicator is operable to display a first visual indicator through an indicator aperture in said trigger housing when said trigger mechanism is in a loaded position, and a second visual indicator when said trigger mechanism is in a released position.

15. The intranasal atomizer according to claim 3 and additionally comprising a drug chamber veil attached to said drug chamber housing;
wherein said atomizer is adapted for single use, and said drug chamber housing and said trigger mechanism are enclosed within said veil and said trigger housing and are not accessible for reloading said drug chamber housing or said trigger mechanism.

16. The intranasal atomizer according to claim 3, wherein said trigger housing additionally comprises at least one breakaway trigger lock tab attached to said trigger button.

17. The intranasal atomizer according to claim 15, wherein said trigger lock tab is operable to prevent activation of said trigger button unless a predetermined minimum trigger activation force is applied to said trigger button.

18. The intranasal atomizer according to claim 1, wherein said drug chamber housing additionally comprises an orifice extending between said hollow drug chamber and said atomizer chamber, and said atomizer comprises an outlet orifice in an outlet end thereof.

19. A method of providing an atomisable drug or medicament, comprising:
providing a sealed drug chamber housing comprising:
a predetermined dose of an atomisable liquid drug or medicament in a hollow drug chamber of said housing;
an atomizer situated at an outlet end of said housing and in fluid communication with said hollow drug chamber;
a plunger sealingly situated within said hollow drug chamber and slidable within said drug chamber; and
a spring operable to drive said plunger within said drug chamber upon release from a compressed position;
providing a safety cap attached to said outlet end of said drug chamber housing; and
providing a trigger housing comprising a trigger button and a trigger mechanism attached to a base flange of said drug chamber housing, wherein said trigger mechanism is operable to retain said spring in a compressed position and to release said spring to drive said plunger upon activation of said trigger button.

20. The method of providing an atomisable drug or medicament according to claim 17, additionally comprising:
removing said safety cap;
placing said outlet end of said drug chamber housing proximate to a nostril of a user; and
activating said trigger button to release said spring and drive said plunger, delivering said atomisable drug or medicament into said nostril of said user.
